(19) United States
(12) Patent Application Publication
(10) Pub. No.: US 2026/0020868 A1
(43) Pub. Date: Jan. 22, 2026

(54) DRILL SHAFT FOR AN INTRAMEDULLARY REAMER, INTRAMEDULLARY REAMER, MOMENTUM-REDUCING COATING, USE OF A POLYETHER ETHER KETONE COATING ON A SHANK, MANUFACTURED FROM NITINOL, OF A DRILL SHAFT OF AN INTRAMEDULLARY REAMER, AND METHOD FOR REPROCESSING AN INTRAMEDULLARY REAMER

(71) Applicant: Peter WITTE, Kiel (DE)

(72) Inventor: Peter WITTE, Kiel (DE)

(21) Appl. No.: 19/268,793

(22) Filed: Jul. 14, 2025

(30) Foreign Application Priority Data
Jul. 18, 2024 (DE) 10 2024 120 414.1

(51) Int. Cl. A61B 17/16; A61B 17/00; A61L 31/02; A61L 31/10

(52) U.S. Cl. CPC A61B 17/164; A61L 31/022; A61L 31/10; A61B 2017/00477; A61B 2017/00526

(57) ABSTRACT

The invention relates to a drill shaft 10 for an intramedullary reamer 1, the intramedullary reamer 1, a momentum-reducing coating 17, the use of said coating 17, and a method for reprocessing the intramedullary reamer 1. The drill shaft 10 includes an elongate shank 19. The shank 19 has a drill connection 13 at a first end 11 and a drive connection 14 at an opposing second end 12. The shank 19 has a shaft core 18 with a longitudinal, end-to-end bore 15. The shaft core 18 is manufactured from nitinol. A lateral surface 16 of the shaft core 18 has a momentum-reducing polyether ether ketone coating 17. The coating 17 extends from the first end 11 to the second end 12.

### PRIORITY CLAIM

This application claims priority to DE 10 2024 120 414.1, filed Jul. 18, 2024.

### BACKGROUND OF INVENTION

### Field of Invention

The invention relates to a drill shaft for an intramedullary reamer. In addition, the invention relates to an intramedullary reamer, to a momentum-reducing coating, and to the use of a polyether ether ketone coating on a shank, manufactured from nitinol, of a drill shaft of an intramedullary reamer.

### Brief Description of Related Art

Intramedullary reamers are often used to drill open the medullary cavity in a patient's bone such that implants can be inserted. In order to reach the region to be drilled open, the drill shafts of such intramedullary reamers are generally designed to be flexible. However, the flexible elements of such drill shafts are often susceptible to germ loads. For this reason, the entire intramedullary reamer needs to be reprocessed and sterilized thoroughly after each use. However, in the region of the flexible drill shaft, in particular, the necessary reprocessing and sterilization is not always possible to the required extent, since germs that are difficult to remove can settle in the gaps between the flexible elements.

One possibility for countering this problem consists in designing the intramedullary reamer as a single-use component and replacing same after each use. However, this incurs high costs and a high material and manufacturing input.

The object of the invention consists in ensuring good reprocessing and sterilization during use of an intramedullary reamer without it being necessary to use excessive amounts of material and without requiring excessively high manufacturing outlay.

### BRIEF SUMMARY OF THE INVENTION

The object is achieved by means of a drill shaft for an intramedullary reamer, comprising an elongate shank, wherein the shank has a drill connection at a first end and a drive connection at an opposing second end, wherein the shank has a shaft core having a longitudinal, end-to-end bore, wherein the shaft core is manufactured from nitinol, wherein a lateral surface of the shaft core has a momentum-reducing polyether ether ketone coating, and wherein the coating extends from the first end to the second end.

A drill shaft made of nitinol has the required material properties, inter alia the required pliability, such that the drill shaft does not need additional flexible elements. However, nitinol has the disadvantage that it splinters if the drill shaft breaks, and the splinters would then enter the patient's body, where they could cause significant damage. To prevent this, the polyether ether ketone coating is provided. Polyether ether ketone is also known as PEEK. On account of a PEEK coating, the splinters of the nitinol shank are prevented from entering the patient's body in the event of a break. The coating therefore acts as a momentum-reducing coating.

The drill shaft is designed as a replacement component. The drill shaft is thus exchanged after every use. However, in order to prevent the costs and the material consumption from being unnecessarily high, only the drill shaft itself is designed as a replacement component. At its first end, the drill shaft has a drill connection for connecting a drill head and, at its second end, has a drive connection for connecting a connecting adapter by means of which the drill shaft can be connected, for example, to a drilling machine. Both the drill head and the connecting adapter are designed such that they can be reprocessed without any problems. As a result, these components can be reused after every use. On account of the drill connection and the drive connection of the drill shaft, the drill head and the connecting adapter can be easily connected to a new drill shaft after being conditioned, in order to thus create a new intramedullary reamer that consists partially of reprocessed parts and partially of a replacement component in the form of the drill shaft.

Preferably, the lateral surface is continuously coated with the polyether ether ketone coating. On account of a continuous coating of the entire lateral surface, splinters are reliably prevented from entering the patient's body. In particular, the first end and the second end are also completely provided with the momentum-reducing polyether ether ketone coating.

Preferably, the coating has a thickness of at least 0.3 mm, in particular at least 0.5 mm. For example, the coating has a thickness of approximately 0.5 mm or approximately 1 mm. Such a layer thickness is sufficient for preventing nitinol splinters from entering the patient's body without unnecessarily restricting the flexibility of the drill shaft.

According to one embodiment, the drill connection has a polygonal connection for a drill head. The polygonal connection is designed, for example, as a hexagon head connection. On account of such a connection in the form of a polygonal connection, the drill shaft can be reliably and securely connected to the drill head.

According to a further embodiment, the drive connection has a polygonal connection for a connecting adapter. The polygonal connection is designed, for example, as a hexagon head connection.

Preferably, the shaft core has a diameter of 6 mm to 15 mm. The shaft core, i.e. the shank without the coating, consists of nitinol, in particular completely. Such a diameter of the shaft core provides the drill shaft with the required flexibility and stability.

Preferably, the bore of the shank has a diameter of 3 mm to 5.5 mm. On account of the bore, the shank is cannulated. A guide wire is drawn through the bore, which securely guides the drill shaft and the drill head during the drilling process and ensures that exactly the planned region of the medullary cavity is drilled open. Together with the use of the guide wire, the cannulation of the intramedullary reamer thus serves to guide the reamer in a directionally stable manner and ensures safe explantation of the intramedullary reamer or an entire intramedullary reamer system.

According to one embodiment, the drill connection and/or the drive connection are designed as a widened portion, wherein the widened portion comprises a circumferential, ramp-shaped surface. Therefore, in the region of the drill connection and/or drive connection, the shank has a wider diameter. Together with the ramp-shaped surface, this serves as a holding surface for a connection of the drill head or else connecting adapter. In particular, the ramp-shaped surface has the shape of a double ramp. In particular, the widened portion has an anti-turn contour. By means of this contour, the drill head or else the connecting adapter, in particular, can be easily mounted on the drill shaft.

This object is also achieved by an intramedullary reamer, comprising a drill shaft according to any one of the embodiments discussed above and a drill head, wherein the drill head has a shank connection that is designed to engage in a form-fitting manner with the drill connection of the drill shaft. The drill head is, in particular, reconditionable. In this way, an intramedullary reamer that comprises both a replaceable drill shaft and a reusable drill head is advantageously provided. In particular, the drill head has a polygonal inner bore which is designed to engage in a form-fitting manner with the polygonal connection of the drill shaft. The drill head has, in particular, a longitudinal, end-to-end bore that is oriented so as to be aligned with the longitudinal bore of the shank when the drill head is secured to the drill shaft.

The intramedullary reamer embodies the same advantages, features, and properties as the drill shaft described above.

Preferably, the intramedullary reamer comprises a connecting adapter, wherein the connecting adapter has a shank connection that is designed to engage in a form-fitting manner with the drive connection of the drill shaft. The drill head and/or connecting adapter are, in particular, releasably securable to the drill shaft. The connecting adapter is, in particular, reprocessable. Thus, the connecting adapter is reusable. In particular, the connecting adapter has a polygonal inner bore that is designed to engage in a form-fitting manner with the polygonal connection of the drill shaft. In particular, the connecting adapter comprises a longitudinal, end-to-end bore that is oriented so as to be aligned with the longitudinal bore of the shank when the connecting adapter is secured to the drill shaft.

According to one embodiment, the intramedullary reamer comprises the drill shaft, the drill connection and/or drive connection of which is designed as a widened portion, wherein the widened portion comprises a circumferential ramp-shaped surface, wherein the intramedullary reamer further comprises at least one sleeve-shaped locking body, wherein the locking body has at least one resilient, inwardly projecting holding element. The ramp-shaped surface or rather surfaces have the shape of a double ramp, in particular. By means of the locking body, the drill head and/or the drive connection can be easily and securely secured to the drill shaft. For this purpose, the resilient, inwardly projecting holding element grips the ramp-shaped surface in order to ensure that the components are securely fitted to one another. The holding element is, in particular, a holding ring. A locking body of this kind is known, for example, from the granted patent DE 10 2019 107 198 B4. In particular, the locking body has at least one anti-turn mechanism. The anti-turn mechanism is, in particular, designed to cooperate in a force-fitting manner with the contour of the widened portion. Preferably, the drill head has a circumferential ramp-shaped surface and, in particular, an anti-turn contour. The connecting adapter also has, in particular, a circumferential, ramp-shaped surface and, in particular, an anti-turn contour. The anti-turn contours of the drill head and connecting adapter are, in particular, each designed to engage in a form-fitting manner with the anti-turn contour or contours of the drill shaft. Preferably, the intramedullary reamer comprises two locking bodies: one for connecting the drill head and one for connecting the connecting adapter.

The object is additionally achieved by means of a momentum-reducing polyether ether ketone coating that is applied to a shaft core, manufactured from nitinol, of a drill shaft of an intramedullary reamer.

In addition, the object is achieved by means of the use of a polyether ether ketone coating on a shaft core, manufactured from nitinol, of a drill shaft of an intramedullary reamer in order to prevent nitinol splinters from entering the patient's body.

The momentum-reducing coating and the use of this coating embody the same advantages, features, and properties as the drill shaft described above and the intramedullary reamer described above.

Finally, the object is achieved by means of methods for reprocessing an intramedullary reamer, wherein a drill head and a connecting adapter are reprocessed and reused, wherein an old drill shaft, which is designed as a single-use replacement part, is replaced and substituted with a new drill shaft, wherein the reprocessed drill head and the reprocessed connecting adapter are combined with the new drill shaft to form a reprocessed intramedullary reamer.

The methods for reprocessing an intramedullary reamer embody the same advantages, features, and properties as the drill shaft described above, the intramedullary reamer described above, the momentum-reducing coating described above, and the use of the coating described above.

According to one embodiment, the old drill shaft and the new drill shaft are in each case a drill shaft according to any one of the embodiments described above and the intramedullary reamer is an intramedullary reamer according to any one of the embodiments described above.

Further features of the invention will become evident from the description of embodiments according to the invention, together with the claims and the appended drawings. Embodiments according to the invention can fulfill individual features or a combination of multiple features.

Within the framework of the invention, features which are labeled with “in particular” or “preferably” are to be understood to be optional features.

### BRIEF DESCRIPTION OF THE DRAWINGS

The invention will be described below, without restricting the general concept of the invention, based on exemplary embodiments and with reference to the drawings, wherein reference is expressly made to the drawings regarding all of the details according to the invention which are not explained in greater detail in the text. In the figures:

FIG. 1 is a schematically simplified perspective exploded representation of an intramedullary reamer having a reconditionable drill head, reconditionable connecting adapter, and replaceable drill shaft;

FIG. 2 is a schematically simplified view of the intramedullary reamer from FIG. 1 in the assembled state;

FIG. 3 is a schematically simplified side view of the intramedullary reamer from FIGS. 1 and 2;

FIG. 4 is a schematically simplified cross-sectional view in the A: A direction; and

FIG. 5 is the cross-sectional view from FIG. 4 without the locking body.

### DETAILED DESCRIPTION OF THE INVENTION

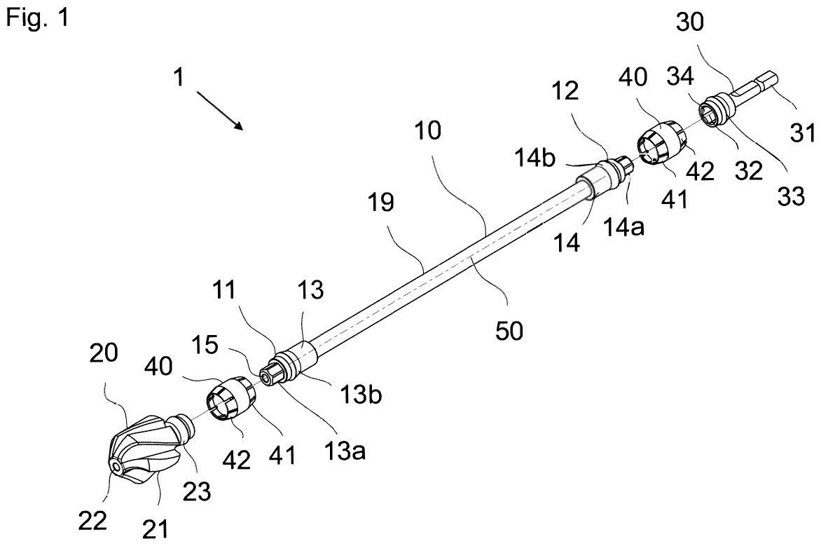

FIG. 1 shows, schematically simplified, an intramedullary reamer 1 in a perspective exploded view. The intramedullary reamer 1 comprises a drill shaft 10, a drill head 20, a connecting adapter 30, and two locking bodies 40. The drill head 20 has a series of cutting edges 21 in order to drill open the medullary cavity in a patient's bone. For this purpose, the drill head 20 also comprises a longitudinal bore 22, which extends all the way through the drill head 20. The bore 22 serves to receive a guide wire (not shown), which ensures that the entire intramedullary reamer 1 can be safely removed after completion of the drilling process. In addition, the bore 22 serves to guide the drill head 20 in a directionally stable manner during the drilling process. Behind the cutting edges 21, the drill head 20 has a hollow-cylindrical portion on which a circumferential ramp-shaped surface 23 is formed, which is provided for securing the drill head 20 to the drill shaft 10. In this embodiment, the ramp-shaped surface 23 has the shape of a double ramp. This portion is thus designed as a shank connection for the drill shaft 10.

The drill shaft 10 is flexible and is designed to absorb the torques applied to the intramedullary reamer 1. For this purpose, a shank 19 of the drill shaft 10 is manufactured from nitinol and provides the drill shaft 10 with the required flexibility and stability. In order to prevent nitinol splinters from entering the patient's body in the event of the shank 19 breaking, a coating consisting of polyether ether ketone (PEEK) is applied to the shaft 19. The drill shaft 10 comprises a drill connection 13 at the first end 11 and a drive connection 14 at the second end 12 in order to connect the drill head 20 and connecting adapter 30, respectively. The drill connection 13 has an anti-turn contour in the form of a polygonal connection 13a, which engages in an anti-turn contour (not visible for perspective reasons) in the form of a polygonal socket in the drill head 20. The drill connection 13 is additionally designed as a widened portion with a greater diameter than the rest of the shank 19 and has a circumferential, double ramp-shaped surface 13b. A sleeve-shaped locking body 40 that has a resilient, inwardly projecting holding element 41, 42 at each of the two ends is provided for securing the drill head 20 to the drill shaft 10. This holding element 41, 42 is, for example, a holding ring. The holding element 41, 42 engages behind the ramp-shaped surfaces 13b, 23 and, in this way, holds the drill head 20 on the drill shaft 10.

The drive connection 14 is designed similarly to the drill connection 13 and has an anti-turn contour in the form of a polygonal connection 14a and a double ramp-shaped surface 14b. Moreover, the connecting adapter 30 has an anti-turn contour in the form of a polygonal inner bore 34, which is designed to engage in a form-fitting manner with the polygonal connection 14a of the drill shaft 10. The outer surface of the connecting adapter 30 additionally comprises a double ramp-shaped surface 33, which forms a shank connection as described above and which cooperates with a holding element 42 of a further locking body 40 in order to secure the connecting adapter 30 to the drill shaft 10.

As in the case of the drill head 20, the drill shaft 10 also has a longitudinal bore 15 and the connecting adapter 30 also has a longitudinal bore 32, which extend in each case entirely in the direction of a longitudinal axis 50 through the components. The intramedullary reamer 1 is thus cannulated. In this way, the guide wire can be drawn through all components 10, 20, 30 in order to guide and remove same. The rear end of the connecting adapter 30 is formed as a machine connection 31 in order to absorb the torque from a drive machine or else drilling machine (not shown).

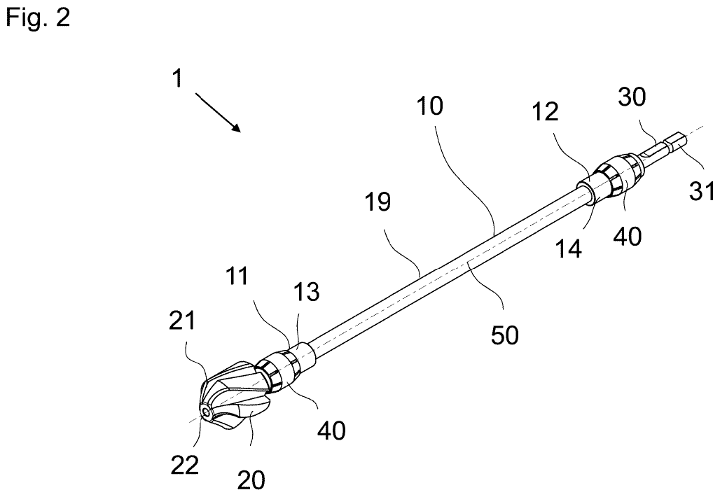

FIG. 2 shows the intramedullary reamer 1 in the assembled state. In this state, the drill head 20 and the connecting adapter 30 are secured to the drill shaft 10 by means of the locking bodies 40. In this way, the resulting intramedullary reamer 1 comprises multiple reconditionable individual parts, namely the drill head 20, the connecting adapter 30, and the locking body 40. Moreover, the intramedullary reamer 1 comprises the single-use drill shaft 10. In this way, difficult and costly conditioning of the drill shaft 10 can be dispensed with without having to replace the entire intramedullary reamer 1. In particular, the drill head 20, which is costly to produce, can be reused in this way without there being the risk of germs settling in difficult-to-reach locations on the drill shaft 10 even in spite of conditioning.

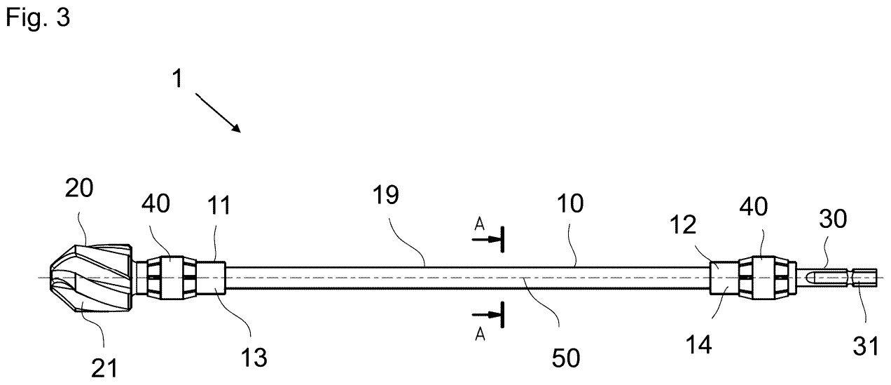

FIG. 3 shows the intramedullary reamer 1 from FIGS. 1 and 2 in a side view. In this view, it becomes clear as to how the drill head 20 and the connecting adapter 30 are fitted on the drill connection 13 and the drive connection 14, respectively, of the shank 19 by means of the locking bodies 40. In addition, a section line A: A is shown, which will be discussed below.

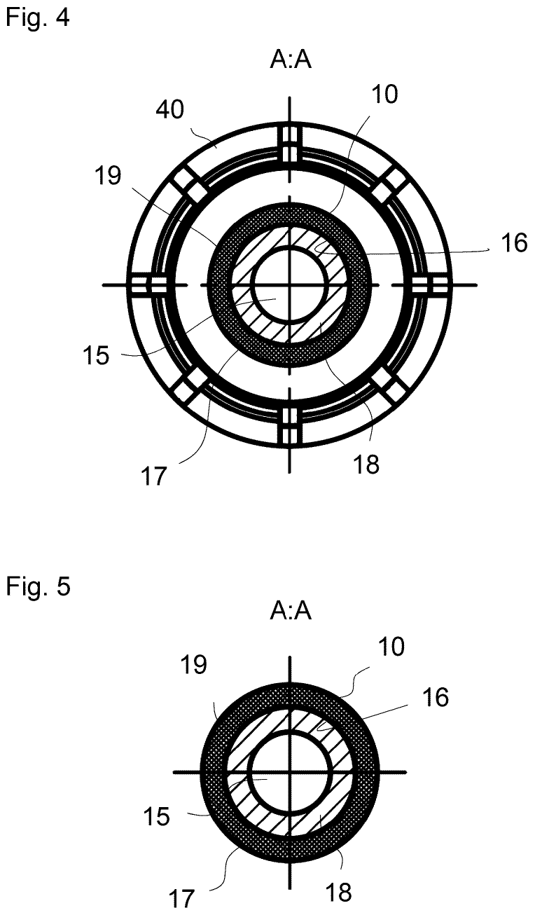

FIG. 4 shows a cross-sectional view along the section plane A: A. Since the viewing direction in this representation is directed towards the connecting adapter 30, the locking body is also visible in FIG. 4, although this is located far behind the actual cross-sectional plane A: A. FIG. 5 therefore shows the view from FIG. 4 without the locking body 40. Both views show that the shank 19 of the drill shaft 10 comprises a nitinol shaft core 18, in which a bore 15 is made centrally. The shaft core 18 is coated along its lateral surface 16 with a momentum-reducing polyether ether ketone (PEEK) coating 17. As outlined above, this coating 17 prevents nitinol splinters of the shaft core 18 from escaping to the outside if the shank 19 breaks.

In the drawings, the same or similar elements and/or parts are, in each case, provided with the same reference signs, and therefore they are not introduced again in each case.

All of the indicated features, including those which are to be inferred from the drawings alone, and individual features which are disclosed in combination with other features, are deemed to be essential to the invention both alone and in combination. Embodiments according to the invention can be fulfilled by individual features or a combination of multiple features.

### LIST OF REFERENCE CHARACTERS

The following reference characters appear in the accompanying drawing figures:
1 Intramedullary reamer
10 Drill shaft
11 First end
12 Second end
13 Drill connection
13a Polygonal connection
13b Ramp-shaped surface
14 Drive connection
14a Polygonal connection
14b Ramp-shaped surface
15 Bore
16 Lateral surface
17 Coating
18 Shaft core
19 Shank
20 Drill head
21 Cutting edge
22 Bore
23 Ramp-shaped surface
30 Connecting adapter
31 Machine connection
32 Bore
33 Ramp-shaped surface
34 Polygonal inner bore
40 Locking body
41 Holding element
42 Holding element
50 Longitudinal axis

What is claimed is:

1. A drill shaft for an intramedullary reamer comprising an elongate shank, wherein the shank has a drill connection at a first end and a drive connection at an opposing second end, wherein the shank has a shaft core having a longitudinal, end-to-end bore, wherein the shaft core is manufactured from nitinol, wherein a lateral surface of the shaft core has a momentum-reducing polyether ether ketone coating, and wherein the coating extends from the first end to the second end.

2. The drill shaft according to claim 1, wherein the lateral surface has a continuous polyether ether ketone coating.

3. The drill shaft according to claim 1, wherein the coating has a thickness of at least 0.3 mm.

4. The drill shaft according to claim 1, wherein the coating has a thickness of at least 0.5 mm.

5. The drill shaft according to claim 1, wherein the drill connection has a polygonal connection for a drill head.

6. The drill shaft according to claim 1, wherein the drive connection has a polygonal connection for a connecting adapter.

7. The drill shaft according to claim 1, wherein the shaft core has a diameter within a range of 6 mm to 15 mm.

8. The drill shaft according to claim 1, wherein the bore of the shank has a diameter within a range of 3 mm to 5.5 mm.

9. The drill shaft according to claim 1, wherein the drill connection has a widened portion, and wherein the widened portion comprises a circumferential, ramp-shaped surface.

10. The drill shaft according to claim 1, wherein the drive connection has a widened portion, and wherein the widened portion comprises a circumferential, ramp-shaped surface.

11. The drill shaft according to claim 10, wherein the drill connection has a widened portion, and wherein the widened portion of the drill connection comprises a circumferential, ramp-shaped surface.

12. An intramedullary reamer comprising a drill shaft according to claim 1 and a drill head, wherein the drill head has a shank connection configured to engage in a form-fitting manner with the drill connection of the drill shaft.

13. The intramedullary reamer according to claim 12, further comprising a connecting adapter, wherein the connecting adapter has a shank connection configured to engage in a form-fitting manner with the drive connection of the drill shaft.

14. The intramedullary reamer according to claim 12, wherein the drill connection of the drill shaft has a widened portion, wherein the widened portion comprises a circumferential, ramp-shaped surface, wherein the drill connection further comprises at least one sleeve-shaped locking body, and wherein the locking body has at least one resilient, inwardly projecting holding element.

15. A method for reprocessing an intramedullary reamer having a drill head, a connecting adapter and an existing drill shaft, which is a single-use replacement part, the method comprising:
reprocessing and reusing the drill head and the connecting adapter; and
replacing and substituting the existing drill shaft with a new drill shaft;
wherein the reprocessed drill head and the reprocessed connecting adapter are combined with the new drill shaft to form a reprocessed intramedullary reamer.

16. The method according to claim 15, wherein the existing drill shaft and the new drill shaft are in each case a drill shaft comprising an elongate shank, wherein the shank has a drill connection at a first end and a drive connection at an opposing second end, wherein the shank has a shaft core having a longitudinal, end-to-end bore, wherein the shaft core is manufactured from nitinol, wherein a lateral surface of the shaft core has a momentum-reducing polyether ether ketone coating, wherein the coating extends from the first end to the second end, and wherein the drill head has a shank connection that is designed configured to engage in a form-fitting manner with the drill connection of the drill shaft.
